(19) United States
(12) Patent Application Publication
(10) Pub. No.: US 2026/0014349 A1
(43) Pub. Date: Jan. 15, 2026

(54) MEDICAL PACKAGING

(71) Applicant: ConvaTec Limited, Flintshire (GB)

(72) Inventors: Alice Young, Chester (GB); Aliaksander Piashevich, Flintshire (GB); Oliver Pfleger, Wirral (GB); Tom Bolton, Salford (GB)

(21) Appl. No.: 19/330,464

(22) Filed: Sep. 16, 2025

Related U.S. Application Data
(63) Continuation of application No. PCT/GB2024/050717, filed on Mar. 15, 2024.
(60) Provisional application No. 63/452,858, filed on Mar. 17, 2023.

(30) Foreign Application Priority Data
Apr. 3, 2023 (GB) 2304951.3

(51) Int. Cl. A61M 25/00; A61J 1/10

(52) U.S. Cl. CPC A61M 25/002; A61M 25/0017; A61J 1/10; A61M 2210/1078

(57) ABSTRACT

A catheter packaging defined by at least two walls bonded along a pair of lateral edges by lateral edge bonds, the packaging having an openable top, wherein the openable top is scaled by a top seal comprising a pair of bond lines which continue from the lateral edge bonds and gradually curve to meet.

### TECHNICAL FIELD OF THE INVENTION

The present invention relates to catheter assemblies. In particular the invention concerns packaged urinary catheter assemblies, and most particularly, but not exclusively packaged intermittent male urinary catheter assemblies, especially “closed” catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

It is desirable that catheter assemblies are discreet such that a layperson may not identify them as a packaged catheter assembly at all. This can make a user feel more comfortable carrying and using them. However, along with being compact, the packaged catheter assembly must remain usable such as by providing visual feedback of the drain of fluid from the body. This can make providing a discreet packaged catheter assembly that is also easy to use difficult and so a user may decide to place the packaged catheter assembly in a dedicated bag to hide it. However, this can cause hygiene issues if the bag is not cleaned/replaced regularly and also increase the weight and bulk of the packaged catheter assembly when carried around.

Users can also have problems in opening packaging and accessing its contents. This can happen because the packaging must be robust enough to ensure its contents is not inadvertently released and so opening of the packaging may require careful manipulation as well as a large force. This can mean the user either cannot use the catheter as they cannot access it or that the package is not opened in a controlled manner. This can cause embarrassment for the user but also inconvenience as the catheter may be rendered useless if it is allowed to fall on the floor or otherwise comes into contact with dirt.

It is an object of embodiments of the present invention to at least partially overcome or alleviate the above problems and/or to provide an improved packaged catheter assembly.

### SUMMARY OF THE INVENTION

In broad terms, the present invention concerns a catheter packaging. In addition, the present invention also concerns a packaged catheter assembly comprising the catheter packaging and a catheter assembly, wherein the packaging is configured to contain the catheter assembly within it.

According to one broad aspect of the present disclosure there is provided a catheter packaging defined by at least two walls. Preferably the two walls are bonded along a pair of lateral edges by lateral edge bonds. Preferably the packaging has an openable top. Preferably the openable top is sealed by a top seal. Preferably the top seal comprises a pair of bond lines. Preferably the pair of bond lines continue from the lateral edge bonds. Preferably the pair of bond lines gradually curve to meet.

As such, according to a first aspect of the present disclosure there is provided a catheter packaging defined by at least two walls bonded along a pair of lateral edges by lateral edge bonds, the packaging having an openable top, wherein the openable top is sealed by a top seal comprising a pair of bond lines which continue from the lateral edge bonds and gradually curve to meet.

Abrupt transitions between the top bond and the lateral edge bonds can cause a stalling of the separation of the two walls as the force pulling apart the two is not aligned with the remaining bonded section, separation at the interface between the top seal and lateral edge seals therefore requires more force, which when still applied after overcoming the interface can result in a lack of control when separating the lateral edges. The provision of top bonds which continue from the lateral edge seals and gradually curve (that is, the interface between the top seal and lateral edge seals is smooth) to meet allows the two walls to be separated in a controlled manner as the force required is constant or nearly constant.

The two walls may comprise a flexible plastics material. For example, polypropylene (PP), polyethylene terephthalate (PET), low density polyethylene (LDPE), metalized polyester (MET PET) or orientated polypropylene (OPP). The two walls may be formed from a single sheet of material.

The lateral edge bond may comprise any one or more of: a weld; mechanical seal; heat seal; pressure seal; adhesive; solvent bond; ultraviolet bond; ultrasonic weld; laser weld; impulse weld; or friction weld. The lateral edge bond may have a width of between 3 and 12 mm, preferably between 4 and 7 mm, more preferably between 4.5 and 5.5 mm, for example 5 mm. The top seal may comprise any one or more of: a weld; mechanical seal; heat seal; pressure seal; adhesive; solvent bond; ultraviolet bond; ultrasonic weld; laser weld; impulse weld; or friction weld. The top seal may have a width of between 3 and 12 mm, preferably between 4 and 7 mm, more preferably between 4.5 and 5.5 mm, for example 5 mm The, or part of the lateral edge bonds may be re-scalable, for example through a resealable adhesive, an additional adhesive that is activated prior or during rescaling, or a hook and loop/hook and hook fastener. The, or part of the top seal may be re-scalable, for example through a resealable adhesive, an additional adhesive that is activated prior or during rescaling, or a hook and loop/hook and hook fastener. The lateral edge bonds and/or top seal may provide a sterile seal. Thus, the lateral edge bonds and/or top seal can be easily constructed to suit the specific needs of the packaging and ensure the contents remains sterile within the packaging.

The lateral edge bonds may form one or more edges of the packaging. A folded edge may comprise the fold between the two walls. All edges other than the folded edge may be formed by bonding. The two walls may comprise a front wall and a rear wall. The first wall may be the front wall. The second wall may be the rear wall. The folded edge may comprise a base of the packaging. The lateral edge bonds may define a left lateral edge and a right lateral edge. The top seal may define a top edge. The left and right lateral edges may be defined as the left and right edges when viewing the packaging in the closed (folded) configuration with the front wall in front of the rear wall, the base at the bottom and the upper edge at the top.

The two walls may be unbonded beyond the top seal. The two walls may be unbonded above the top seal. The two walls may extend beyond the top seal. A portion of each wall above the top seal may form a flap. A portion of each wall beyond the top seal may form a flap. There may be two flaps. A distal end of the flap may define an upper edge of the packaging. One or both flaps may be provided with a finger hole. The finger hole may extend through the respective wall. Both finger holes may be arranged above the point where the pair of bond lines meet. Both finger holes may be sized to allow a user's finger to be at least partially inserted through. Both finger holes may be a circle, an oval, an ellipse, a polygon, a square, a rounded square, a stadium, a triangle, a rounded triangle. Preferably both finger holes have a rounded triangular shape. The finger hole may taper towards the top seal. The width of the finger hole may decrease towards the top seal. Where the finger hole is triangular (or a rounded triangular shape) the vertex may be aligned with the point where the two bond lines meet.

Accordingly, in a preferred embodiment of the present disclosure, there is provided a catheter packaging defined by at least two walls bonded along a pair of lateral edges by lateral edge bonds, the packaging having an openable top, wherein the openable top is sealed by a top seal comprising a pair of bond lines which continue from the lateral edge bonds and gradually curve to meet, wherein the two walls are unbonded beyond the pair of bond lines, the two falls forming two flaps, wherein the finger holes have a rounded triangular shape.

In a more preferrable embodiment of the present disclosure, there is provided a catheter packaging defined by at least two walls bonded along a pair of lateral edges by lateral edge bonds, the packaging having an openable top, wherein the openable top is sealed by a top seal comprising a pair of bond lines which continue from the lateral edge bonds and gradually curve to meet, wherein the two walls are unbonded beyond the pair of bond lines, the two falls forming two flaps, wherein the finger holes have a rounded triangular shape, wherein the finger holes taper towards the top seal.

The finger holes may have a width defined as the distance between a point closest to the left lateral edge and a point closest to the right lateral edge of at least 20, 30, 40, 50 mm. The finger holes may have a width of no more than 60, 50, 40, 30 mm. Preferably the width of the finger hole is between 15 and 30 mm, most preferably between 15 and 25, for example 20 mm. The width of the finger holes may be between 10% and 30% the width of the packaging, preferably between 15% and 25% the width of the packaging, for example 20%.

The packaging may have a width defined as the distance between the left lateral edge and right lateral edge of the packaging of at least 70, 80, 90 or 100 mm. The packaging may have a width of no more than 200, 180, 160, 140 or 120 mm. Preferably, the width is between 90-120 mm, more preferably between 100 and 110 mm and most preferably about 105 mm.

The packaging may have a height defined as the distance between the upper edge and base of at least 160, 170, 180, 190 or 200 mm. The packaging may have a height of no more than 300, 290, 280, 270 or 260 mm. Preferably, the height is between 200-275 mm, and most preferably it is about 220 mm. Thus, the packaging is a compact shape when folded so that it is convenient and discreet for the user to carry, for example it is easier to fit in a bag or pocket.

The lateral edge bonds may comprise a frangible portion. The frangible portion of the lateral edge bonds may be proximate to the top seal. The lateral edge bond may comprise a tear stop. The tear stop may delimit the frangible portion of the lateral edge bonds from a non-frangible portion of the lateral edge bonds. The tear stop may comprise a widened portion of the lateral edge bond. The tear stop may be at least twice as wide as the lateral edge bond. The tear stop may have a width of at least 5 mm, 7 mm 10 mm or 12 mm. The tear stop may have a width of between 5 and 15 mm, preferably between 7 and 15 mm, more preferably between 10 and 15 mm, for example between 11 and 13 mm, such as 12 mm. The tear stop may be bonded with a stronger bonding technique than the frangible portion of the lateral edge bond. The tear stop may require a separation force at least twice the separation for the frangible portion of the lateral edge bond. The top seal may be frangible.

A longitudinal axis may be defined as extending along the centre of the packaging between the upper edge and base. A transverse axis may be defined as perpendicular to the longitudinal axis, extending between the right lateral edge and left lateral edge. The packaging may comprise a centreline extending between the top seal and base midway between the left lateral edge and right lateral edge.

The pair of bond lines may meet at a point. By meeting at a point, that is an apex, the bond lines have a tapering point at their uppermost edge, this means the first point to be separated has a very small cross section thereby reducing the force needed to initiate the separation. The pair of bond lines may meet midway between the lateral edges. The pair of bond lines may meet at a centreline. The pair of bond lines may by symmetrical. Each bond line may comprise a single curve. Each bond line may comprise a single continuous curve. Each bond line may comprise a first curved portion and a second straight portion. The first curved portion may be adjacent to the lateral edge bond. The second straight portion may be adjacent to the meeting point of the pair of bond lines. The bond lines may be straight where they meet. The (first) curved portion may comprise at least 30%, at least 35%, at least 40%, at least 45%, at least 50%, of the length of the respective bond line. Each bond line may comprise a first curved portion and a second curved portion. The first curved portion may curve towards the centreline. The curved portion may have a radius of curvature of at least 10, 20, 30, 40, 50 mm. Preferably the curved portion has a radius of curvature of between 10 and 70 mm, more preferably between 20 and 50 mm, such as between 30 and 40 mm, for example 35 mm. The curved portion may have a radius of curvature of at least 10%, 20%, 30%, 40% of the width of the packaging. Preferably the curved portion has a radius of curvature of between 10% and 50% of the width of the packaging, more preferably between 20% and 40%, such as between 30% and 40%, for example between 30 and 35%, such as 33%. The curved portion may have a radius of curvature at least 3 times, 4 times, 5 time, 6 times, the width of the bond line. Preferably, the curved portion has a radius of curvature between 3 and 10 times the width of the bond line, more preferably between 4 and 9 times the width of the bond line, such as between 5 and 8 times the width for the bond line, for example 7 times the width of the bond line. A bond line which the above degree of curvature provides for the gradual transition between the top seal and the lateral edge seal, providing a smoother and more consistent separation.

The finger holes and the top seal may have a separation of no more than 30 mm, 20 mm, 15 mm, 12 mm, 10 mm, 8 mm. The finger holes and the top seal may have a separation of at least 1 mm, 2 mm, 4 mm, 6 mm. The finger holes and the top seal may have a separation of between 4 and 20 mm, preferably between 6 and 15 mm, more preferably between 7 and 12 mm, for example 8 mm.

The packaging may be a sterile packaging. That is when sealed the packaging provides a sterile cavity within.

The packaging may contain a catheter assembly. The catheter assembly may comprise a catheter. The catheter assembly may comprise a fluid collection bag.

Accordingly, in a second aspect of the disclosure there is provided a packaged catheter assembly comprising a catheter and a fluid collection bag arranged in catheter packaging, the packaging defined by at least two walls bonded along a pair of lateral edges by lateral edge bonds, the packaging having an openable top, wherein the openable top is sealed by a top seal comprising a pair of bond lines which continue from the lateral edge bonds and gradually curve to meet.

The catheter packaging of the second aspect may be the catheter packaging of the first aspect and may include any optional feature thereof.

The catheter assembly is preferably a closed catheter assembly, that is that the catheter assembly comprises a catheter coupled to a fluid collection bag, such that it is configured to collect and retain fluid released from the body through the catheter. The fluid collected/catheter assembly may then be disposed of by the user when is convenient. Of course, embodiments may also be open catheter assemblies in that fluid is not collected or retained by the catheter assembly.

According to a third aspect of the disclosure there is provided a catheter packaging, defined by two walls bonded along a pair of lateral edges, the packaging having an openable top, wherein the openable top is sealed by a top seal, wherein the two walls are unbonded above the top seal to form flaps, one or both flaps comprising a finger hole, wherein the finger hole has a rounded triangular shape which tapers towards the top seal.

The top seal may comprise a pair of bond lines. The bond lines may continue from the lateral edge bonds and gradually curve to meet.

The catheter packaging of the third aspect may comprise any of the optional features of the first aspect.

According to a fourth aspect of the disclosure there is provided a catheter packaging defined by two walls bonded along a pair of lateral edges, the packaging having an openable top, wherein the openable top is sealed by a top seal; where the top seal comprises two angled bond lines which meet at a point.

At least one of the two angled bond lines may have a straight portion and a curved portion. The bond lines may continue from the lateral edge bonds and gradually curve to meet.

The packaging of the fourth aspect may comprise any of the optional features of the packaging of the first aspect.

### DETAILED DESCRIPTION OF THE INVENTION

In order that the invention may be more clearly understood one or more embodiments thereof will now be described, by way of example only, with reference to the accompanying drawings, of which:

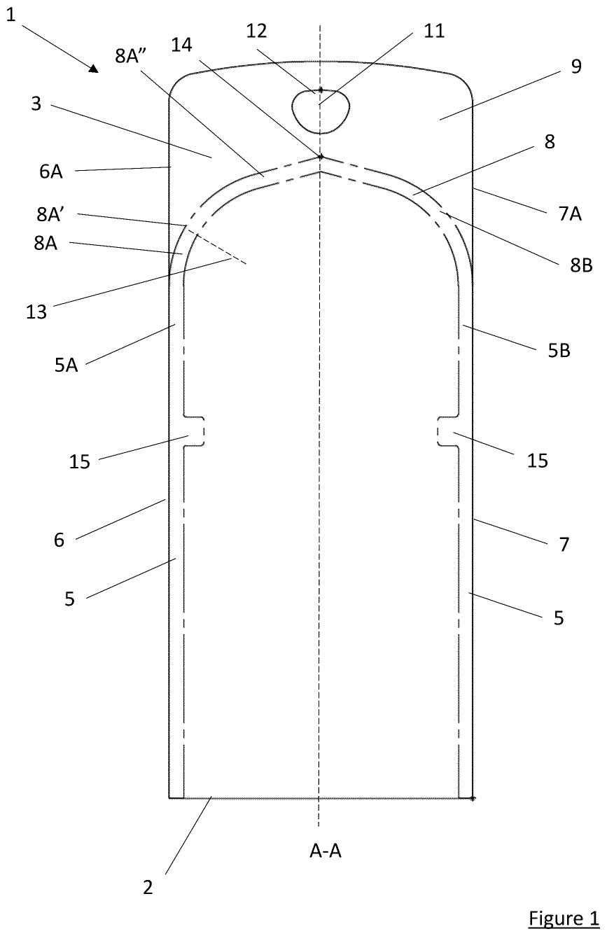

FIG. 1 is a front view of a first embodiment of a catheter packaging;

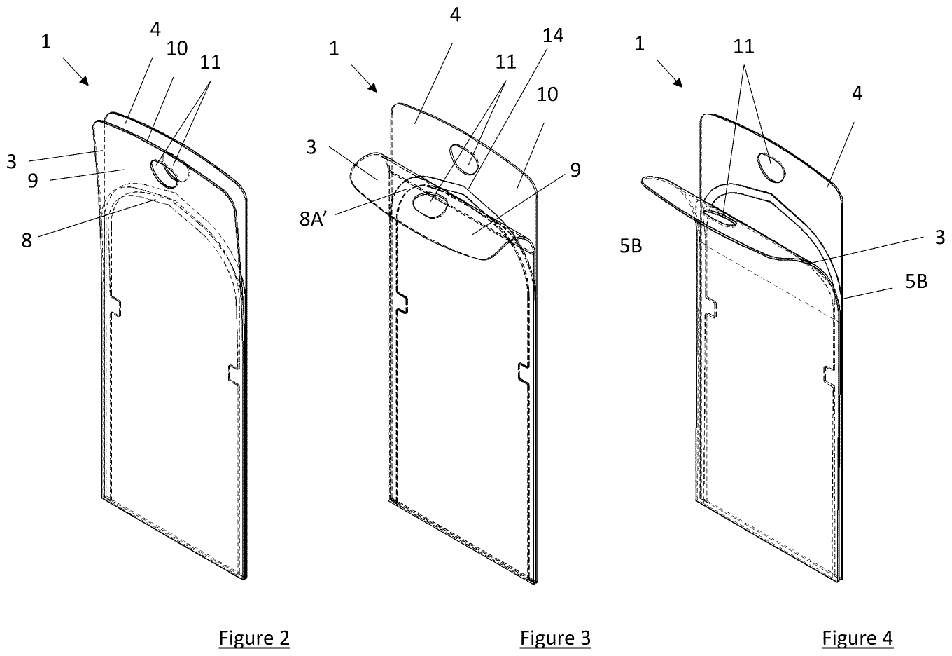

FIG. 2 is a perspective view of the catheter packaging of FIG. 1 in a closed configuration;

FIG. 3 is a perspective view of the catheter packaging of FIG. 1 where the top seal is broken and the bond lines are being separated;

FIG. 4 is a perspective view of the catheter packaging of FIG. 1 where the top seal bond lines are separated and the opening is transitioning to the lateral edge bonds; and

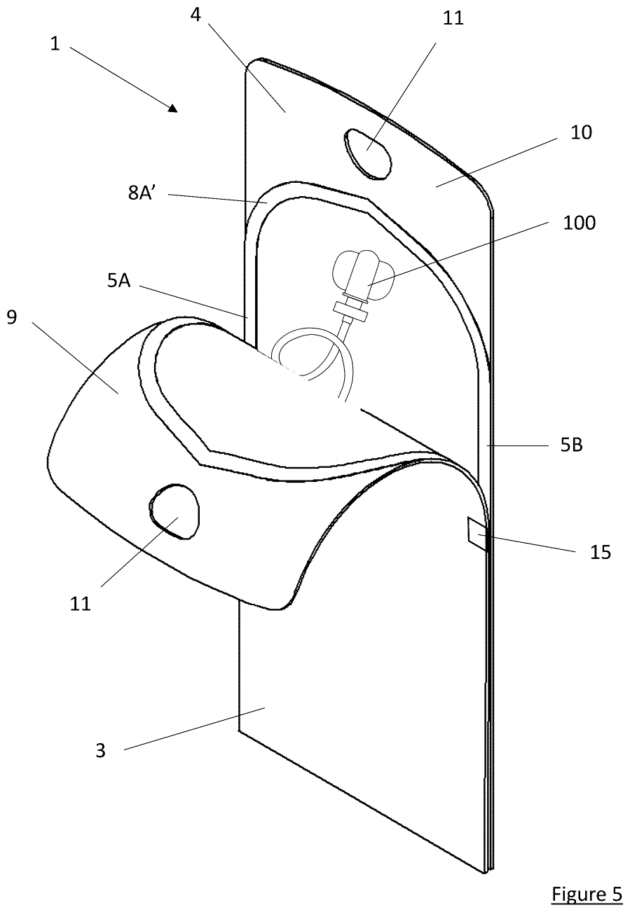

FIG. 5 is a perspective view of the catheter packaging of FIG. 1 in an open configuration and showing a catheter assembly contained within.

In the figures, as is conventional, broken lines show hidden features.

Referring to FIGS. 1 and 2, a first embodiment of a catheter packaging 1 is shown. In this embodiment the packaging 1 is formed from a single piece of material. The packaging 1 is formed by folding this single piece of material along a fold-line 2 spanning the width of the packaging at a point midway along its length to form a front wall 3 and rear wall 4 of the packaging. Lateral edge bonds 5 bond the front 3 and rear walls 4 along their lateral edges and a top seal 8 is provided between the two lateral edge bonds opposite the fold line 2. To aid in the description of the packaging, a longitudinal axis can be considered to extend along a centreline A-A of the packaging from the fold line 2 to the top seal 8.

In this embodiment, the packaging fold-line 2 forms a base edge 2 of the packaging. In this embodiment, the lateral edge bonds 5 form a left lateral edge and a right lateral edge. The left lateral edge 6 and right lateral edge 7 being defined as the right and left sides of the packaging 1 when viewing the packaging 1 with the rear wall 4 behind the front wall 3, the base edge 2 at the bottom of the packaging 1 and the top seal 8 at the top of the packaging 1.

In this embodiment, lateral edge bonds 5 and top seal 8 are formed through heat sealing the front wall 3 to the second wall 4 with a bond width of X mm, but in other embodiments any suitable bonding may be used such as Velcro®, chemical adhesives, chemical bonding, ultrasonic bonding, or mechanical bonding. It is also conceivable that no folds may be provided, for example the front 3 and rear 4 walls may be provided separate sheets of material, in in place of a fold line two walls 3,4 may also be joined along the base edge 2 with heat scaling or other suitable bonding.

In this embodiment, the top seal 8 and a portion of the lateral edge bonds are configured to break to allow the packaging 1 to be opened. Consequently, the packaging 1 has a closed configuration in which it is sealed, and an open configuration in which an openable top is open and a catheter assembly within is accessible.

In this embodiment, the packaging 1 is formed from an opaque material and as such provides a discreet package for the packaged catheter assembly which can make the user more comfortable in carrying the packaged catheter assembly in day-to-day life. In addition, the exterior of the packaging 1 may have a textured or printed appearance and feel, such as a matte finish to reduce the impression that it is a medical device.

In this embodiment, a portion of the lateral edges 6,7 of the front and rear walls 3,4 are unbonded beyond the top seal 8. The unbonded portion of the front wall 3 forms a front opening tab 9 and the unbonded portion of the rear wall 4 forms a rear opening tab 10. Both the front opening tab 9 and rear opening tab 10 project from the packaging 1 along an edge corresponding to the top seal 8. Each of the tabs 9, 10 of this particular embodiment are rectangular in shape with lateral edges 6A, 7A parallel to and continuing from a respective lateral edge 6,7 of the packaging. At a midpoint of each tab 9,10 there is provided a finger hole 11. Both finger holes 11 have a rounded triangular shape which tapers towards the top seal 8, the finger hole 11 may have a width, measured along the base 12 of 3 cm, and a height, measured from the base to the apex 13 of the taper of 2.5 cm. The width of the finger holes 11 thereby less than 20% if the width of the packaging. By providing finger holes 11 which taper towards top seal 8 the force applied to the finger holes 11 to break the top seal 8 is more directly focused to the top seal 8, this effect is particularly prevalent where the finger holes 11 have a width of no more than 25% of the width of the packaging. The narrowness of the finger hole thereby reducing the force required and making the packaging 1 easier to open.

The top seal 8 may be provided by two bond lines 8A,B, each bond line is a continuation of one of the lateral edge bonds 5, that is a left bond line 8A is a continuation of a left lateral edge bond 5A and a right bond line 8B is a continuation of a right lateral edge bond 5B.

In this embodiment the left bond line 8A and right bond line 8B are congruent, one being the reflection of the other about the centreline A-A, as such only the left bond lone 8A will be described in detail, and the skilled person will be able to infer the properties of the right bond line 8B accordingly.

As noted above the left bond line 8A is a continuation of the left lateral edge bond 5A, and the interface between the two is smooth, the left bond line 8A gradually curves away from the left lateral edge 6 with a radius of curvature 13 of approximately 30 mm, at a point along the left bond line 8A approximately half way between the left later edge 6 and the centreline A-A the left bond line 8A continues to the centreline A-A in a straight line. Thus, the left bond line 8A has a first curved portion 8A′ and a second straight portion 8A″. At the centreline the left bond line 8A and right bond line 8B meet at an angle, and thus form a point 14.

In other embodiments the bond lines 8A,B may only have a curved portion 8A′, the curve being suitably shaped so that the two bond lines 8A,B meet at the centreline A-A. Where the bond lines are formed only of a single curve, they may meet at a point, or alternatively be shaped so as to form a single continuous curved line.

It is particularly beneficial to the opening of the packaging that the tapering of the finger hole 11 is aligned with the point 14 (where present) as this helps direct the force of opening to a small initial opening as will be described in detail below. Whilst in this embodiment the finger holes 11 and point 14 are provided on the centreline, it will be appreciated that by providing the right and left bond lines 8A,B with different profiles the point of meeting could be varied across the width of the packaging 1.

The lateral edge bonds 5 may each be provided with a tear stop 15, the tear stops are arranged approximately two-thirds of the way along the lateral edge bonds 5 from the base 2 and comprise a reinforced portion of the lateral edge bond 5. In this embodiment the reinforcement is provided by increasing the width of the lateral edge bond 5, alternatively the tear stops 15 could be reinforced in other ways, for example a high temperature weld could be used or an adhesive applied. The position of the tear stops 15 determined the extent to which the cavity can be opened and the degree of access to the catheter assembly inside. In some embodiments the tear stops 15 are provided closer to the base 2, this improves the access the contents of the packaging, but also increases the risk the contents may accidentally spill. Conversely, the tear stops 15 could be provided further from the base, this would reduce the risk of accidentally dropping the contents, but also hinder their removal.

The base 2, lateral edge bonds 5 and top seal 8 together define a sterile cavity within the packaging in which a catheter assembly (not shown) can be stored prior to use.

With reference to FIGS. 2 to 5 the opening of the packaging 1 is described. When the user wishes to use the catheter assembly 100 they put a finger (or thumb) into each of the finger holes 11 and gripping the tabs 9,10 pull the two tabs 9,10 apart, as shown in FIG. 3. As a result of the tapered finger holes 11 being aligned with the point 14 of the two bond lines 8A,B the force required to initiate the separation of the front 3 and rear 4 walls is reduced. This is because the force is directed by the shape of the finger holes 11 to the point 14 and the seal at tip of the point 14 requires the least force to break.

Continued pulling apart of the two tabs 9,10 continues the breaking of the top seal 8 along the bond lines 8A,B, the gradual curve of the curved section 8A′ means that the separation of the bond lines 8A,B does not meet a discontinuity which would otherwise cause the separation to snag and thereby require a greater force to proceed, as such the curved sections 8A′ provide for a smooth opening with a reduction in force required to separate the two walls 3,4 and also in the variation in force required during the opening. Having transitioned around along the curved sections 8A′ the separation continues along the lateral edge bonds 5A,B as shown in FIG. 4.

The separation of the front 3 and rear 4 walls continues until the lateral edge bonds 5A,B are separated up to the tear stops 15. Due to the increased width of the lateral edge bonds 5 at the tear stops 15 an increased force is required to separate the lateral edge bonds 5 beyond this point. The increased resistance provides tactile feedback to the user that the packaging 1 has been opened to a sufficient degree. The user can now access the catheter assembly 100 within the packaging for use. Once the user has used the catheter assembly it may be returned to the packaging for later disposal. Where the top seal 8 and lateral edge bonds 5 comprise a resealable bond, the packaging may be resealed around the used catheter assembly.

The one or more embodiments are described above by way of example only. Many variations are possible without departing from the scope of protection afforded by the appended claims.

1. A catheter packaging defined by at least two walls bonded along a pair of lateral edges by lateral edge bonds, the packaging having an openable top, wherein the openable top is sealed by a top seal comprising a pair of bond lines which continue from the lateral edge bonds and gradually curve to meet.

2. A catheter packaging according to claim 1 wherein the pair of bond lines meet at a point.

3. A catheter packaging according to claim 1 or 2 wherein the pair of bond lines meet at a centreline.

4. A catheter packaging according to any preceding claim wherein the pair of bond lines are symmetrical.

5. A catheter packaging according to any preceding claim wherein at least one of the bond lines comprises a first curved portion and a second straight portion.

6. A catheter packaging according to claim 5 wherein the first curved portion is adjacent to the lateral edge bond and the second straight portion is adjacent to the meeting point of the pair of bond lines.

7. A catheter packaging according to claim 5 or 6 wherein the first curved portion comprises at least 30% of the length of the respective bond line.

8. A catheter packaging according to any preceding claim wherein at least one of the bond lines has a radius of curvature of at least 20 mm.

9. A catheter packaging according to any preceding claim wherein at least one of the bond lines has a radius of curvature of at least 30 mm.

10. A catheter packaging according to any preceding claim wherein at least one of the bond lines has a radius of curvature of between 10% and 40% of the width of the packaging.

11. A catheter packaging according to any preceding claim wherein at least one of the bond lines has a radius of curvature of between 20% and 40% of the width of the packaging.

12. A catheter packaging according to any preceding claim wherein at least one of the bond lines has a radius of curvature of between 30% and 40% of the width of the packaging.

13. A catheter packaging according to any preceding claim wherein at least one of the bond lines has a radius of curvature at least 3 times the width of the bond line.

14. A catheter packaging according to any preceding claim wherein the two walls are unbonded beyond the pair of bond lines, the two walls forming two flaps.

15. A catheter packaging according to claim 14 wherein each flap is provided with a finger hole extending through the wall.

16. A catheter packaging according to claim 15 wherein the finger holes are arranged above the point where the pair of bond lines meet.

17. A catheter packaging according to claim 15 or 16 wherein the finger holes have a rounded triangular shape.

18. A catheter packaging according to claim 17 wherein the finger holes taper towards the top seal.

19. A catheter packaging according to any of claims 15 to 18 wherein the width of the finger holes is between 15% and 25% the width of the packaging.

20. A catheter packaging according to any preceding claim wherein the packaging is a sterile packaging.

21. A catheter packaging according to any preceding claim wherein the lateral edge bonds comprise tear stops.

22. A catheter packaging according to claim 21 wherein the tear stops comprise a widened portion of the lateral edge bond.

23. A catheter packaging according to claim 22 wherein the lateral edge bonds comprise a frangible portion and a non-frangible portion and the tear stop delimits the frangible portion and non-frangible portion.

24. A packaged catheter assembly comprising a catheter and a fluid collection bag arranged in catheter packaging, the catheter packaging defined by at least two walls bonded along a pair of lateral edges by lateral edge bonds, the packaging having an openable top, wherein the openable top sealed by a top seal comprising a pair of bond lines which continue from the lateral edge bonds and gradually curve to meet.

25. A packaged catheter assembly according to claim 24 wherein the catheter is a closed catheter.

26. A packaged catheter assembly according to claims 24 and 25 wherein the catheter packaging is a catheter packaging according to any of claims 1 to 23.
